(19) United States
(12) Patent Application Publication
(10) Pub. No.: US 2008/0021520 A1
(43) Pub. Date: Jan. 24, 2008

(54) SYSTEM FOR THE TRANSCUTANEOUS STIMULATION OF A NERVE IN THE HUMAN BODY

(75) Inventor: Stefan DIETRICH, ERLANGEN (DE)

Correspondence Address: LUCAS & MERCANTI, LLP, 475 PARK AVENUE SOUTH, 15TH FLOOR, NEW YORK, NY, 10016, US

(73) Assignee: CERBOMED GMBH, ERLANGEN (DE)

(21) Appl. No.: 11/779,130

(22) Filed: Jul. 17, 2007

(30) Foreign Application Priority Data
Jul. 18, 2006 (DE) 10 2006 033 623.2

(51) Int. Cl. A61N 1/02

(52) U.S. Cl. 607/59

(57) ABSTRACT

The invention relates to a system (1) for the transcutaneous stimulation of a nerve in the human body which comprises an apparatus (2) which has at least one stimulation electrode (3) and at least one, reference electrode (4) for transcutaneous nerve stimulation, where the at least one stimulation electrode (3) and the at least one reference electrode (4) are connected to a control unit (5) and can receive an electric current therefrom, the at least one stimulation electrode (3) and the at least one reference electrode (4) being arranged in or on a housing (6) which is designed to be fitted on, in or behind the human ear. In order to increase the functionality of the system, the invention provides that the system (1) also has a programming appliance (7) which can be used to define the variation for at least one stimulation parameter (I, U, t, f) for the nerve stimulation, where the apparatus (2) also has a programmable control element (8) having a memory element (9) which is connected to the control unit (5) of the apparatus (2) and which can be used to store a program which includes the variation for at least one stimulation parameter (I, U, t, f), with at least one temporary communication link (10, 10′, 10″) being set up between the programming appliance (7) and the control element (8) for the purpose of transmitting information, particularly data for the stimulation parameter (I, U, t, f), between the programming appliance (7) and the control element (8).

The invention relates to a system for the transcutaneous stimulation of a nerve in the human body which comprises an apparatus which has at least one stimulation electrode and at least one reference electrode for transcutaneous nerve stimulation, where the at least one stimulation electrode and the at least one reference electrode are connected to a control unit and can receive an electric current therefrom, the at least one stimulation electrode and the at least one reference electrode being arranged in or on a housing which is designed to be fitted on, in or behind the human ear.

It is generally known practice to use invasive and non-invasive stimulation of the nerves to influence their neurophysiological and neuroelectrical quality and hence the function of the stimulated nerves. This allows various pathological conditions to be treated. There are numerous apparatuses intended both for invasive and non-invasive stimulation. The initial basis for the invention presented here is the method of transcutaneous electrical nerve stimulation. This method involves applying pulsed currents with various pulse shapes, amplitudes, pulse durations and frequencies through the skin to various nerves and altering the status parameters thereof in an advantageous manner.

A transcutaneously operating nerve stimulation apparatus of the type mentioned at the outset is known from DE 10 2005 003 735 A1, for example.

In the case of the solutions known to date, the following circumstances have been found to be disadvantageous:

The stimulation parameters need to be set on the appliance itself, resulting in an increased space requirement for electrical components. This is usually done using small potentiometers which are put into the desired position using a miniature screwdriver.

This makes an apparatus of this kind larger and less comfortable to wear. Wearers of such apparatuses, particularly for treating depression, want a design which is as inconspicuous and small as possible, since this avoids a certain stigmatization effect.

It is not possible to fix the parameter settings absolutely. This can result in unwanted setting alterations to the detriment of the appliance's therapeutic benefit. In addition, the patient or wearer of the apparatus is provided with the option of making setting alterations independently, which is often not desired from a therapeutic point of view.

It has been found that the lack of a memory element and of an appropriate connection to a programming appliance makes it impossible to store data about stimulation parameters, data about the stimulation (e.g. duration, time, date) or product information on the appliance and to read or alter these when required.

For widespread therapeutic use of the method, it is desirable to develop a system which takes account of these requirements.

The invention is based on the object of developing a system of the type mentioned at the outset such that this desire is satisfied. The aim is to provide a system for transcutaneous nerve stimulation which allows a stimulation apparatus worn on, in or behind the ear to be connected to a programming unit by means of a wired or wireless link. The connection needs to be able to be used to send a wide variety of stimulation parameters, for example in the form of stimulation algorithms. In addition, data also need to be able to be read from a storage medium in the stimulation apparatus.

The way in which this object is achieved by the invention is characterized in that the system cited at the outset is distinguished in that the system also has a programming appliance which can be used to define the variation for at least one stimulation parameter for the nerve stimulation, where the apparatus also has a programmable control element having a memory element which is connected to the control unit of the apparatus and which can be used to store a program which includes the variation for at least one stimulation parameter, with at least one temporary communication link being set up between the programming appliance and the control element for the purpose of transmitting information, particularly data for the stimulation parameter, between the programming appliance and the control element.

In this case, the basic apparatus for nerve stimulation is designed particularly for stimulating the vagus nerve in the region of the external auditory canal and/or the auricle. To achieve optimum positioning of the at least one stimulation electrode and of the at least one reference electrode, the apparatus is preferably designed such that the housing has a bow-shaped extension which is designed for positioning an electrode head in the region of the external auditory canal and/or the auricle. The bow-shaped extension may imitate the shape of the external auditory canal and/or of the auricle.

One subject matter of the invention presented here is the option of using the stimulation apparatus to generate a wide variety of stimulation parameters such as the frequency, the pulse width, the pulse duration or amplitude. To this end, the apparatus has a programmable memory element which may either be in the form of a standalone component, e.g. in the form of a flash or RAM, or is represented as an integrated region on a microprocessor. This is used to store algorithms which depict the various settings of the stimulation parameters and use various other circuits, e.g. a pulse generator, to prompt current to be applied to the stimulation electrode. Such a memory can be used to store not only information and algorithms relating to the stimulation parameters but also other data, such as patient data or information from at least one sensor which is integrated in the apparatus.

For programming, an external programming appliance is proposed which can be used to define the variation for at least one stimulation parameter. One development provides for the use of a commercially available PC for this purpose which has software for a parameter and data definition installed on it. The programming appliance may also comprise a dedicated housing with a display and control elements.

Between the programming appliance and the stimulation apparatus, there is at least temporarily a connection to ensure that information is interchanged back and forth. The connection may be a wireless (e.g. Bluetooth, IR, DECT, ZigBee) or wired (e.g. USB) type of connection.

In one development, the parameters set by an authorized person (e.g. by a doctor) can no longer be changed by the wearer of the apparatus, that is to say by the patient. Alternatively, provision may be made for the treating doctor to provide the patient with the option of setting at least one stimulation parameter as desired or in a particular value range himself. This can be done using a thumbwheel or a multifunction switch, for example.

To allow a doctor to obtain information regarding the stimulation process retrospectively, for example, provision is made for the stimulation apparatus to be equipped with a clock (real-time clock or timer). In combination with other components, e.g. a microprocessor or a flash, this may allow checking while the appliance is being worn. To this end, the appliance is equipped not only with the primary current source, for example in the form of a rechargeable storage battery, but also with at least one auxiliary current source.

One development provides for a charging station associated with the stimulation apparatus for recharging the integrated storage battery to be developed such that contact elements firstly allow the current source to be charged and secondly allow data interchange between the stimulation apparatus and the programming appliance. This data interchange can be effected using electrical contact elements or else using optically (e.g. infrared), audio (e.g. Bluetooth) or other media, for example.

Preferably, the charging station, the programming unit and the stimulation appliance are in tune with one another via particular circuits such that during data transmission or the charging process it is not possible to apply a stimulation current to the stimulation electrode of the stimulation apparatus, which achieves a high level of patient safety.

The drawing shows an exemplary embodiment of the invention, where:

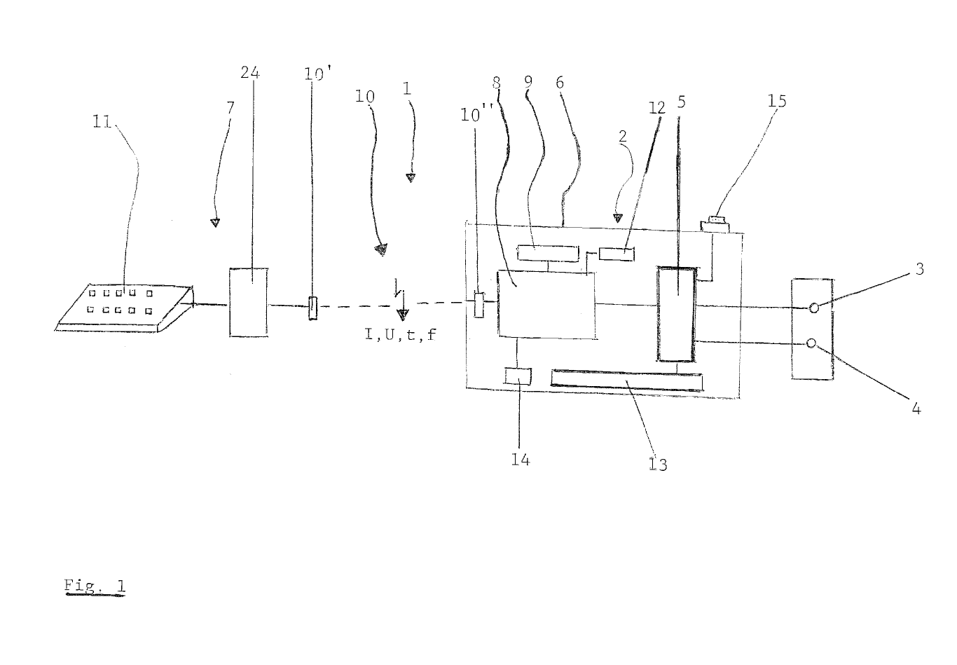

FIG. 1 schematically shows the circuit for a stimulation apparatus for the transcutaneous stimulation of the vagus nerve and the connection to a programming appliance, and

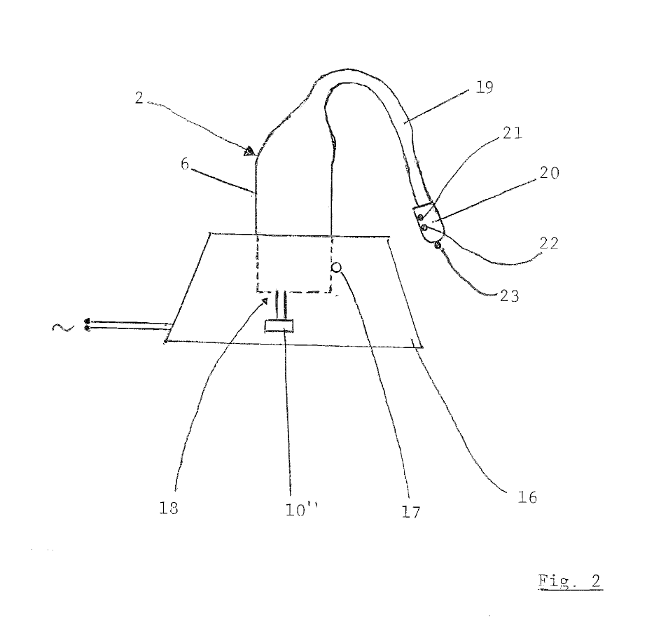

FIG. 2 schematically shows the stimulation appliance, designed as a BTE (Behind The Ear) appliance, which is in a charging station.

FIG. 1 shows the circuit diagram for a system 1 for the transcutaneous stimulation of a nerve. In particular, the auricular area of the vagus nerve is stimulated in order to influence psychovegetative parameters. This allows physical and mental stress to be removed, for example, or depression or other neuropsychiatric disorders can be positively influenced.

The apparatus 2 for the transcutaneous stimulation of the vagus nerve essentially comprises a programmable control element with a stimulation circuit 8, a memory element 9, an electronic clock 12, a control unit 5, a first power supply (e.g. battery/storage battery) 13 for supplying power to the primary circuit, a second current source (battery/storage battery) 14 as an auxiliary power supply, at least one setting means 15 and a stimulation electrode 3 and a reference electrode 4. The components of the apparatus 2 are accommodated in a housing 6.

The apparatus 2 can be connected to a programming appliance 7 by means of a communication link 10, comprising the communication modules 10′ and 10″. The programming appliance has a PC 24 and also a data input apparatus 11 (keyboard).

One portion 10″ of the communication link 10 is in the housing 6 of the apparatus 2, and the other portion 10′ is connected to the PC 24.

The communication link 10 can be used to load various stimulation parameters, such as current level (I), voltage (U), time period (t) and frequency (f), onto the apparatus 2 and particularly into its memory element 9 or to retrieve data from the apparatus 2.

FIG. 2 schematically shows the apparatus 2 for transcutaneous nerve stimulation in a charging station 16. The housing 6 of the apparatus 2 is plugged into the charging station 16. There, it can be connected to a communication link 10″, for example, by means of various contact elements 18. In addition, means 17 are provided for preventing a stimulation current so long as the apparatus 2 is in the charging station 16. These means 17 prevent current from being applied to contact points 21 and 22. The contact points 21, 22 are arranged in an electrode head 20 which is in turn arranged at the end of a bow-shaped extension 19.

In addition, a sensor 23 may be accommodated in the electrode head 20—as can be seen in the exemplary embodiment.

The proposed system 1 allows a particularly advantageous mode of operation for the apparatus 2 for the transcutaneous stimulation of the vagus nerve:

The doctor who has the programming appliance 9 can use the communication link 10 to make contact with the apparatus 2 and, by way of example, to transmit data for the treatment from the programming appliance 9 to the apparatus 2 and particularly to the memory element 9. These data are particularly the aforementioned parameters I, U, t and f.

In this context, the individual parameters may be provided with information regarding whether and, if so, to what extent the patient himself can change the parameters by making a setting on the apparatus 2. For this, it is possible, on a parameter-specific basis, to define value ranges within which the patient himself can make a setting. Other parameters can be excluded from being able to be set by the patient if this is not recommended for the treatment.

### LIST OF REFERENCE SYMBOLS

1 System for the transcutaneous stimulation of a nerve

2 Apparatus

3 Stimulation electrode

4 Reference electrode

5 Control unit

6 Housing

7 Programming appliance

8 Programmable control element with a stimulation circuit

9 Memory element

10 Communication link

10′ Communication link

10″ Communication link

11 Data input apparatus

12 Electronic clock

13 First battery

14 Second battery

15 Setting means

16 Charging station

17 Means for preventing a stimulation current

18 Contact element

19 Bow-shaped extension

20 Electrode head

21 Contact point

22 Contact point

23 Sensor

24 PC

I, U, t, f, Stimulation parameter

I Current

U Voltage

t Time period

f Frequency

1. System for the transcutaneous stimulation of a nerve in the human body which comprises an apparatus which has at least one stimulation electrode and at least one reference electrode for transcutaneous nerve stimulation, where the at least one stimulation electrode and the at least one reference electrode are connected to a control unit and can receive an electric current therefrom, the at least one stimulation electrode and the at least one reference electrode being arranged in or on a housing which is designed to be fitted on, in or behind the human ear, wherein the system also has a programming appliance which can be used to define the variation for at least one stimulation parameter (I, U, t, f) for the nerve stimulation,
where the apparatus also has a programmable control element having a memory element which is connected to the control unit of the apparatus and which can be used to store a program which includes the variation for at least one stimulation parameter (I, U, t, f),
with at least one temporary communication link being set up between the programming appliance and the control element for the purpose of transmitting information, particularly data for the stimulation parameters (I, U, t, f), between the programming appliance and the control element.

2. System according to claim 1, wherein the programming appliance, the communication link and the control element are designed such that all the relevant stimulation parameters (I, U, t, f) for stimulating a nerve can be transmitted from the programming appliance to the control element.

3. System according to claim 1 wherein, the apparatus also has setting means which can be used to influence a defined portion of the stimulation parameters (I, U, t, f), particularly for the person wearing the apparatus.

4. System according to claim 3, wherein the setting means are designed such that the stimulation parameters (I, U, t, f) which can be influenced using the setting means can be influenced only in prescribed value ranges.

5. System according to claim 1, wherein the control unit, the control element and the memory element are designed such that the memory element can be used to store the stimulation parameters (I, U, t, f) assigned to the user of the apparatus.

6. System according to claim 1, wherein the apparatus also comprises an electronic clock which is connected to the control element.

7. System according to claim 1, wherein the communication link comprises a connecting wire which can be arranged between the programming appliance and the apparatus.

8. System according to claim 1, wherein the communication link is in the form of a wireless link.

9. System according to claim 8, wherein that the wireless link is a radio link, particularly a Bluetooth link, a WLAN link, a DECT link or a ZigBee link.

10. System according to claim 1, wherein the programming appliance comprises a data input apparatus, particularly a keyboard.

11. System according to claim 1, wherein the programming appliance comprises a PC.

12. System according to claim 1 wherein the apparatus also has a first battery for supplying power particularly to the control unit of the apparatus.

13. System according to claim 1, wherein the apparatus also has a second battery for supplying power particularly to the control element of the apparatus.

14. System according to claim 12, wherein it also has a charging station for charging at least the first battery.

15. System according to claim 14, wherein the apparatus and/or the charging station are provided with means which prevent a stimulation current from being produced on the electrodes of the apparatus when the apparatus is in the charging station.

16. System according to claim 14, wherein the charging station has an element for setting up the communication link, where contact elements are provided which are used to set up a connection between the apparatus and the element for setting up the communication link when the apparatus is in the charging station.

17. System according to claim 1, wherein the apparatus has a bow-shaped extension which is designed for insertion into the auditory canal, where the bow-shaped extension imitates the shape of the entrance to the auditory canal or of the external auditory canal, and where there is, at the end of the bow-shaped extension, an electrode head which has two contact points for the two electrodes.

18. System according to claim 17, wherein that the electrode head is designed and suitable for stimulating the vagus nerve in the region of the external auditory canal and/or the auricle.

19. System according to claim 17 wherein the electrode head is made of a soft material, particularly soft silicone.

20. System according to claim 1, wherein the apparatus also has at least one sensor for measuring a physiological parameter for the person using the apparatus.
